(19) United States
(12) Patent Application Publication
(10) Pub. No.: US 2025/0322912 A1
(43) Pub. Date: Oct. 16, 2025

(54) SEED SEQUENCE GENERATION METHOD AND APPARATUS FOR ITD ANALYSIS IN NGS ANALYSIS

(71) Applicant: THE CATHOLIC UNIVERSITY OF KOREA INDUSTRY-ACADEMIC COOPERATION FOUNDATION, Seoul (KR)

(72) Inventors: Myung-Shin KIM, Seoul (KR); Yong-Goo KIM, Seoul (KR); Jong-Mi LEE, Seoul (KR); Insik HWANG, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 18/862,573

(22) PCT Filed: Apr. 28, 2023
(86) PCT No.: PCT/KR2023/005884

§ 371 Date: Nov. 4, 2024

(30) Foreign Application Priority Data
May 2, 2022 (KR) 10-2022-0054353

(51) Int. Cl. G16B 40/10; C12Q 1/6869; G16B 30/10

(52) U.S. Cl. CPC G16B 40/10; C12Q 1/6869; G16B 30/10

(57) ABSTRACT

One embodiment of the present invention relates to a method comprising: acquiring information about reads for an arbitrary sequence by means of an NGS analysis method; selecting reads having the same insertion sequence from among the acquired reads on the basis of a reference sequence, and b) selecting reads having the same soft-clipped bases; and selecting, as a SEED, a region including a part or all of the sequence of the soft-clipped bases of the selected reads and the insertion sequence thereof, and thus ITD can be accurately analyzed through the selected SEED, such that diagnosis, prognosis determination and the like of diseases associated with ITD can be performed thereby.

### TECHNICAL FIELD

The present disclosure relates to SEED generation method and apparatus for deriving ITD in NGS analysis, and more particularly, to a method and an apparatus for selecting a SEED to easily distinguish ITD from a read sequence derived from NGS analysis.

### BACKGROUND ART

Currently, an NGS test has been performed worldwide in medical settings to diagnose genetic diseases, and research in the field of precision medicine has been actively conducted through the NGS test. NGS technology used in precision medicine variously includes panel sequencing, exome sequencing, whole genome sequencing, and the like. Although NGS enables rapid and accurate sequencing of genes, there is a problem in that accurate ITD analysis is difficult due to the limitations of NGS analysis when analyzing internal tandem duplication (ITD) using NGS.

To solve the problems of ITD analysis in NGS analysis, several commercial analysis programs have been introduced, but ITD analysis still shows limitations, and the present disclosure was invented to solve the problems of commercial analysis programs.

### DISCLOSURE

### Technical Problem

An object of the present disclosure is to provide a method and an apparatus for deriving a SEED to facilitate ITD analysis in order to quickly and accurately analyze ITD.

### Technical Solution

According to an aspect of the present disclosure, there is disclosed a method for deriving a sequence for analyzing internal tandem duplication (ITD) in an NGS method, the method comprising: 1) acquiring reads by an NGS method: 2) a) selecting reads having the same insertion sequence from among the acquired reads based on a reference sequence: or/and b) selecting reads having the same soft-clipped bases: and 3) selecting, as a SEED, a region including a part or all of the sequence of the soft-clipped bases of the selected reads or/and the insertion sequence thereof.

According to an exemplary embodiment of the present disclosure, in the selecting of the reads in step 2), when three or more reads have the same insertion sequence, the reads having the same insertion sequence may be selected.

According to another exemplary embodiment of the present disclosure, in the selecting of the reads in step 2), when three or more reads have the same sequence of the soft-clipped bases, the reads may be selected.

According to an exemplary embodiment of the present disclosure, in step 3), a region including the sequence of the soft-clipped bases may include an adjacent sequence from the 3′ or 5′ end of the soft-clipped base, in which the sequence length including the adjacent sequence from the 3′ or 5′ end of the soft-clipped base may be 12 bp to 20 bp.

According to another exemplary embodiment of the present disclosure, in step 3), a region including the insertion sequence may include an adjacent sequence from the 3′ or 5′ end of the insertion sequence, in which the sequence length including the adjacent sequence from the 3′ or 5′ end of the insertion sequence may be 12 bp to 20 bp.

According to an exemplary embodiment of the present disclosure, the NGS method may be an amplicon-based NGS method.

According to another aspect of the present disclosure, there is disclosed a method for analyzing internal tandem duplication (ITD) in an NGS method, the method comprising:
1) acquiring reads by an NGS method:
2) a) selecting reads having the same insertion sequence from among the acquired reads based on a reference sequence: or/and b) selecting reads having the same soft-clipped bases:
3) selecting a region including a part or all of the sequence of the soft-clipped bases or/and insertion sequence of the selected reads as a SEED: and
4) analyzing sequences matched with the SEED with respect to reads acquired by an arbitrary NGS method using the selected SEED as a query.

According to an exemplary embodiment of the present disclosure, the analyzing of step 4) may be a step of counting the number of matched sequences.

According to yet another aspect of the present disclosure, there is disclosed an apparatus for deriving a sequence for analyzing internal tandem duplication (ITD) in next generation sequence (NGS) analysis, the apparatus including: a processor configured to acquire information about reads for an arbitrary sequence by an NGS analysis method, select reads having the same insertion sequence from among the acquired reads based on a reference sequence; or/and b) select reads having the same soft-clipped bases, and select, as a SEED, a region including a part or all of the sequence of the soft-clipped bases of the selected reads or/and the insertion sequence thereof: a memory configured to store information about the reads and information about the reference sequence and the SEED; and a display configured to display information about the derived SEED.

According to an exemplary embodiment of the present disclosure, in the selecting of the reads, when three or more reads have the same insertion sequence, the reads having the same insertion sequence may be selected.

According to another exemplary embodiment of the present disclosure, in the selecting of the reads, when three or more reads have the same sequence of the soft-clipped bases, the reads may be selected.

According to an exemplary embodiment of the present disclosure, a region including the sequence of the soft-clipped bases may include an adjacent sequence from the 3′ or 5′ end of the soft-clipped base, in which the sequence length including the adjacent sequence from the 3′ or 5′ end of the soft-clipped base may be 12 bp to 20 bp.

According to another exemplary embodiment of the present disclosure, a region including the insertion sequence may include an adjacent sequence from the 3′ or 5′ end of the insertion sequence, in which the sequence length including the adjacent sequence from the 3′ or 5′ end of the insertion sequence may be 12 bp to 20 bp.

### Advantageous Effects

According to an exemplary embodiment of the present disclosure, the method or the apparatus can derive a SEED capable of rapidly and accurately performing specific ITD analysis from reads acquired by an NGS method to rapidly and accurately derive the state and number of ITDs from NGS reads of a patient from the derived SEED. Therefore, it is possible to monitor a disease condition of the patient using the SEED.

### DESCRIPTION OF DRAWINGS

FIG. 1 is a schematic diagram for describing a method for deriving a SEED according to an exemplary embodiment.

FIG. 2 is a diagram confirming an effect of ITD analysis using a SEED according to one exemplary embodiment.

FIG. 3 is a diagram illustrating an example of analyzing reads using a SEED derived from the present disclosure on IGV.

FIG. 4 is a flowchart for describing a method for deriving a SEED according to an exemplary embodiment.

FIG. 5 is a flowchart for describing a method for deriving a SEED according to an exemplary embodiment in more detail.

FIG. 6 is a block diagram of an apparatus according to an exemplary embodiment.

### BEST MODE OF THE INVENTION

Terms used in the present specification will be described in brief and the present disclosure will be described in detail.

Terms used in the present disclosure adopt general terms which are currently widely used as possible by considering functions in the present disclosure, but the terms may be changed depending on the intention of those skilled in the art, precedents, emergence of new technology, etc. Further, in a specific case, there are terms arbitrarily selected by an applicant, and in this case, the meanings of the terms will be disclosed in detail in a corresponding description part of the present disclosure. Accordingly, the terms used in the present disclosure should be defined based on not just names of the terms but the meanings of the terms and the contents throughout the present disclosure.

Throughout the specification, when a certain part “comprises” a certain component, unless explicitly described to the contrary, it will be understood to further include other components, but not the exclusion of other components. In addition, terms including “unit”, “module”, and the like disclosed herein mean a unit that processes at least one function or operation, which may be implemented by hardware or software or a combination of hardware and software.

As used in the present invention, the term “next-generation sequencing” or “NGS” refers to any sequencing method that determines one nucleotide sequence of either individual nucleic acid molecules (e.g., in single molecule sequencing) or clonally expanded proxies of individual nucleic acid molecules in a high throughput manner (e.g., 10, 100, 1000 or more molecules are sequenced simultaneously). The next-generation sequencing method is known in the art and described, for example, in Metzker, M. (2010) Nature Biotechnology Reviews 11:31-46. The next-generation sequencing may detect variants that are present in less than 5% of nucleic acids in a sample.

As used in the present invention, the term “amplicon-based NGS method” refers to a technology that designs primers capable of amplifying a target gene to produce various short-length reads, and then sorts and analyzes the short-length reads, and representative technology includes an Emulsion PCR method, and devices based thereon include 454 platform of Roche, SOLid platform and Ion Torrent platform of Thermo Fisher, etc. NGS using the amplicon method has an advantage of low library complexity and fast analysis speed compared to a probe-based hybridization method. Amplicon-type NGS data contains primer sequences in a leading sequence of the reads. This primer sequence is designed to have the same sequence as the standard sequence.

### (1) Selection of Target

A target sequencing method is generally as follows. To find a causal gene of a disease, using next-generation sequencing, the whole genome may be sequenced, only an exome region may be targeted and sequenced, or a specific gene may also be targeted and sequenced. Sequencing only the exome region or specific target gene is advantageous in terms of cost and efficiency. In addition, since genetic changes often result in direct diseases such as cancer, detecting changes in base sequence in the exome region or target gene may be effective in finding the causal gene. To sequence only the exome or target gene, a library capable of capturing only the exome or target gene is required.

### (2) Massively Parallel DNA Sequencing

Next Generation Sequencing (NGS) may perform sequencing faster and in a larger scale at one time than conventional capillary sequencing, and an amplification process of a sample using a vector used in the conventional capillary sequencing is omitted, and thus there is an advantage in that it is possible to avoid experimental errors occurring in the amplification process.

NGS systems produced by three companies have been mainly used. The Roche 454 GS FLX launched in 2004 was the first introduced NGS equipment, and the equipment performs sequencing using a pyrosequencing method and an emulsion polymerase chain reaction to identify specific bases based on the intensity of light emitted at the final step of the experiment. When operated for 7 hours, a sequence of about 100 Mb may be identified, which has significantly higher performance than a conventional ABI 3730 device that may identify a sequence of 440 kb in the same time.

The Illimina Genome Analyzer from Illumina introduces the concept of sequencing by synthesis, which attaches single-stranded DNA fragments to a glass plate and then polymerizes the fragments to form clusters. When going through this process, sequencing is performed while identifying types of bases attached to a DNA fragment to be tested, and 40 to 50 million fragments with a length of 32 to 40 bases are produced by an operation for about 4 days.

A Sequencing by Oligo Ligation (SOLID) device from Life Technologies attaches DNA fragments to be tested to 1 μm-sized magnetic beads and then performs sequencing using an emulsifier-polymerase chain reaction. When performing the sequencing, a method of repeatedly attaching 8-mer fragments is used, and bases to be used for actual sequencing are located at positions 4 and 5 of 8-mer. The remaining portion attached after the positions is linked with a fluorescent material, which indicates which base will complementarily bind to the DNA fragment to be tested. By attaching all 8-mers five times in one binding cycle and performing the same operation five times, a sequence of a DNA fragment consisting of a total of 25 bases may be identified. The feature of the SOLID device is sequencing using two-base encoding, and the method is to confirm the same region through twice sequencing when determining one base sequence. The sequencing is performed while moving the sequence by one base per one binding cycle toward an adapter attached to the magnetic beads. This process has an advantage of eliminating errors that occur during a sequencing experiment.

### (3) Analysis of Sequencing Data

In order to find the causal gene of the disease, it is necessary to investigate what changes have occurred from a conventional gene base sequence, so that sequencing data (sequence reads) of an individual (patient) are compared with a reference genome or reference sequence. This operation is referred to as mapping. After finding out a difference between the individual and the reference genome through mapping, appropriate selection criteria are set to extract only reliable base sequence variation information (variant calling). The variation information is structural variation (SV) including single nucleotide variation (SNV), short insertion/deletion (Short Indel), copy number variation (CNV), fusion genes, and the like. Then, the base sequence variation information is compared with a conventional database to determine whether the variation has already been discovered or is newly discovered. In addition, it is expected whether the variation will lead to a change in amino acids or what effect the variation will have on a protein structure. This process is referred to as annotation. Information about extracted single nucleotide variants and short insertions/deletions may be listed in a database to further improve the quality of information, or research may also be conducted to find disease-causing variants through integrated research with a genome-wide association study (GWAS).

As used in the present disclosure, the term “acquire” or “acquiring” refers to obtaining possession of a physical entity or value, for example a numerical value, by “directly acquiring” or “indirectly acquiring” the physical entity or value. The “indirectly acquiring” means performing a process for obtaining the physical entity or value (e.g., performing a synthetic or analytical method). The “indirectly acquiring” refers to receiving a physical entity or value from another party or source (e.g., a third-party laboratory that directly acquired the physical entity or value).

The indirectly obtaining of the physical entity includes performing a process including a physical change in a physical material, such as a starting material. Representative changes include making a physical entity from two or more starting materials, shearing or fragmenting a material, isolating or purifying a material, combining two or more separate entities into a mixture, or performing a chemical reaction including breaking or forming covalent or non-covalent bonds. The indirectly acquiring of the value includes performing a treatment including a physical change in a sample or another material, for example, performing an analytical process that includes a physical change in a material, such as a sample, an analyte, or a reagent (sometimes, referred to in the present specification as a “physical analysis”), and performing an analysis method including, for example, one or more methods below: separating or purifying a material, for example, an analyte or a fragment thereof or other derivatives thereof, from another material; combining the analyte or fragment thereof or other derivatives thereof with another material, such as a buffer, a solvent or a reactant; changing the structure of the analyte or fragment thereof or other derivatives thereof, for example by breaking or forming a covalent or non-covalent bond between a first atom and a second atom of the analyte; or changing the structure of a reagent or fragment thereof or other derivatives thereof, for example by breaking or forming a covalent or non-covalent bond between a first atom and a second atom of the reagent.

As used in the present disclosure, the term “acquiring the sequence” or “acquiring the reads” is used in the present specification and refers to obtaining possession of a nucleotide sequence or an amino acid sequence by “directly acquiring” or “indirectly acquiring” the sequence or reads. The “directly acquiring” of the sequence or reads means performing a process for obtaining a sequence (e.g., performing a synthesis or analysis method), such as performing a sequencing method (e.g., a next-generation sequencing (NGS) method). The “indirectly acquiring” of the sequence or reads refers to receiving a sequence, or information or knowledge of the sequence, from another party or source (e.g., a third-party laboratory that acquires the sequence directly). The acquired sequence or reads need not be a complete sequence, and obtaining information or knowledge that identifies one or more alterations disclosed in the present specification, such as sequencing at least one nucleotide or being present in a subject, constitutes acquiring a sequence.

The directly acquiring of the sequence or reads includes performing a process including a physical change in a physical material, for example, a starting material, such as a tissue or cell sample, for example a biopsy or an isolated nucleic acid (e.g. DNA or RNA) sample. Representative changes include shearing or fragmenting two or more starting materials, such as preparing physical entities from genomic DNA fragments (e.g., isolating a nucleic acid sample from tissue); combining two or more separate entities into a mixture, and performing a chemical reaction that includes breaking or forming covalent or non-covalent bonds. The directly acquiring of the value includes performing a process including a physical change in a sample or another material as described above.

As used in the present disclosure, the term “nucleic acid” or “polynucleotide” means deoxyribonucleic acid (DNA) or ribonucleic acid (RNA) and polymers thereof in a single-stranded or double-stranded form. Unless otherwise specifically limited, the term includes nucleic acids containing known analogues of natural nucleotides that have similar binding properties to a reference nucleic acid and are metabolized in a manner similar to natural nucleotides. Unless otherwise stated, a specific nucleic acid sequence also implicitly includes conservatively modified variants (e.g., degenerate codon substitutions), alleles, orthologs, SNPs, and complementary sequences thereof, in addition to an explicitly disclosed sequence. Specifically, the degenerate codon substitutions may be achieved by generating sequences in which position 3 of one or more selected (or all) codons is substituted with mixed bases and/or deoxyinosine residues. The term nucleic acid is used interchangeably with genes, cDNA, mRNA, small noncoding RNA, micro RNA (miRNA), Piwi-interacting RNA, and short hairpin RNA (shRNA) encoded by a gene or locus.

As used in the present disclosure, the term “paired-end read” means that ‘paired end’ refers to both ends of the same DNA molecule. When one end is sequenced, and then reversed and the other end is sequenced, these two ends having identified base sequences are called ‘paired-end read.’ For example, Illumina sequencing generates reads of about 500 bps and reads 75 bps of a base sequence from both ends of the read. At this time, reading directions of the two reads (a first read and a second read) are 3′ and 5′, which are opposite to each other and become paired-end reads to each other.

As used in the present disclosure, the term “soft-clip”, “soft-clip segment” or “soft clipped read” means a read in which only some of reads acquired from NGS are mapped to a reference genome (reference sequence) and the rest is not mapped.

As used in the present disclosure, the term “soft-clip bases” refers to unmatched sequences that exist after the end of a matched portion after matching the reference sequence in the soft clipped read.

As used in the present disclosure, the term “brick point” means the end of a sequence that is only partially mapped to the reference genome (reference sequence) in the “soft clipped read”.

As used in the present disclosure, the term “insertion sequence” means a sequence additionally inserted in a read, compared to the reference sequence (base sequence).

As used in the present disclosure, the term “disconcordant read pair” means that a read pair (a first read and a second read) acquired by paired end read sequencing is not mapped on the same reference gene, but is mapped on different positions or different chromosomes.

As used in the present disclosure, the term “concordant read pair” means having information that the read pair (the first read and the second read) acquired by paired end read sequencing has been mapped on the same gene, but a soft clip segment portion of the read is mapped to another gene.

As used in the present disclosure, the term “SEED” refers to a sequence derived in the present invention to perform ITD analysis quickly and accurately.

### MODES OF THE INVENTION

Hereinafter, the present disclosure will be described in more detail through exemplary embodiments. However, these exemplary embodiments are more specifically illustrative the present disclosure, and the scope of the present disclosure is not limited to these exemplary embodiments.

According to an exemplary embodiment of the present disclosure, there is provided a method for deriving a SEED for rapid and accurate ITD analysis in NGS analysis for a specific target sequence.

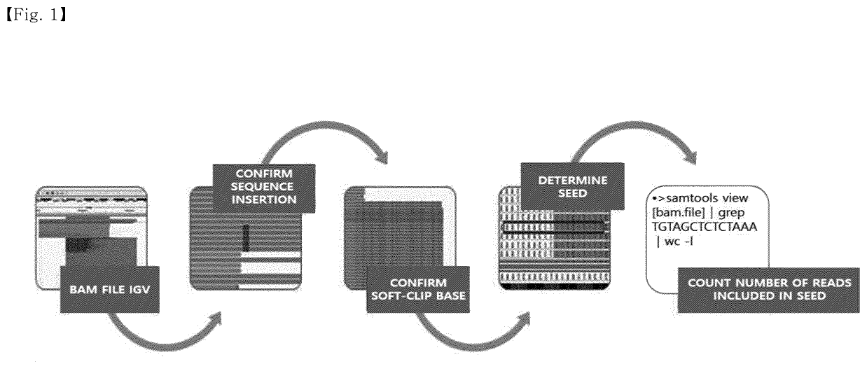

Referring to FIG. 1, the method for deriving the SEED according to an exemplary embodiment may be performed by loading a BAM file generated by an amplicon method into an Integrative Genomics Viewer (IGV), setting a maximum downsized read count to 10,000, performing sort alignment of reads by an insertion size to confirm whether three or more reads have the same insertion sequence, performing sort alignment of the reads by a base to confirm whether three or more reads have the same sequence of soft-clipped bases, and then determining a SEED of 8 to 30 bp, preferably 12 to 20 bp along the boundary of the insertion sequence or soft-clipped bases using the confirmed sequence. Thereafter, the number of reads including the determined SEED may be counted using a samtool command and divided by the total count to determine a variant allele frequency (VAF).

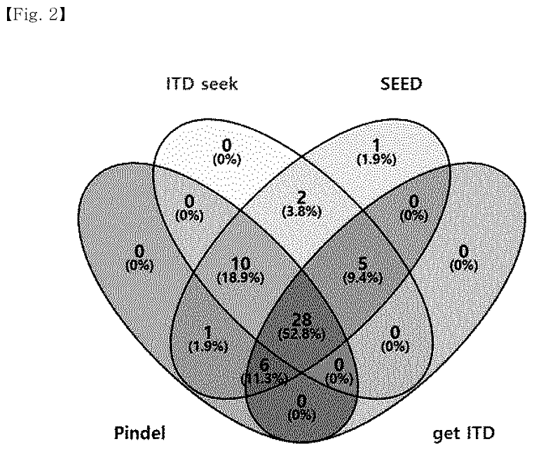

FIG. 2 is a diagram of comparing results of analyzing ITD using a SEED derived by an exemplary embodiment with results of analyzing ITD using another method. Specifically, simulations were performed for each method based on 53 known NGS read information and ITD information.

As illustrated in FIG. 2, when a total of 53 ITDs were analyzed, the method of the present disclosure found all ITDs, but other methods could only find some thereof.

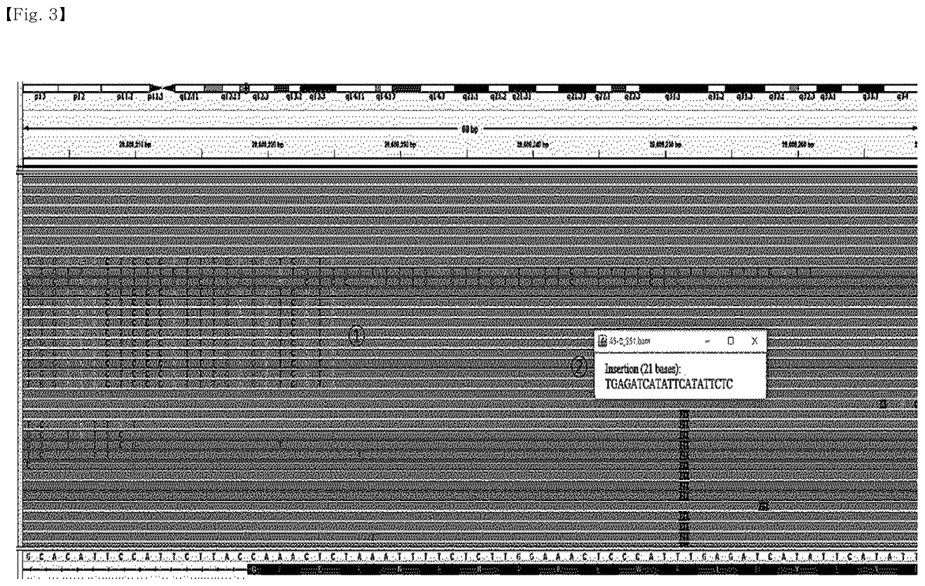

FIG. 3 is an example of ITD analysis performed using a SEED derived according to an exemplary embodiment.

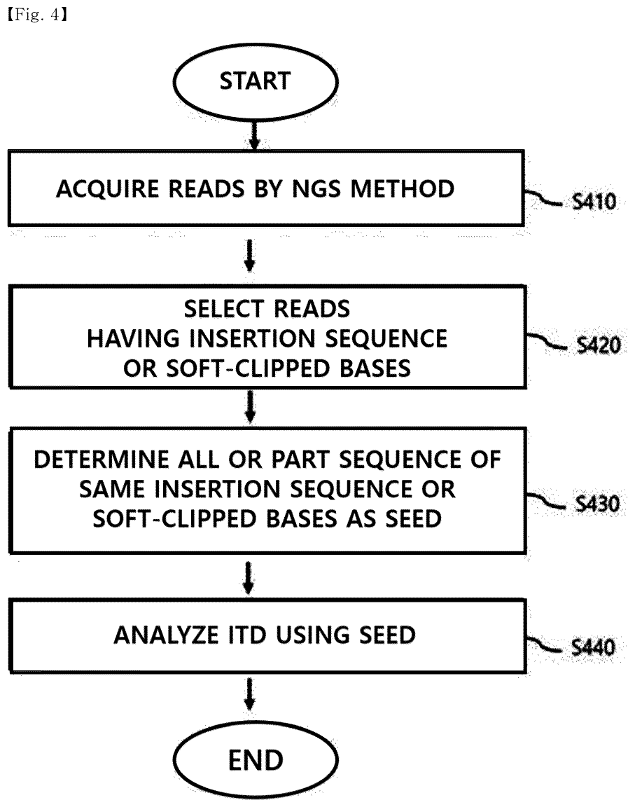

FIG. 4 is a flowchart for describing a method for deriving a SEED according to an exemplary embodiment.

In step S410, reads of a target region may be acquired from the genome of a subject or from previously stored data. To obtain the reads, various NGS methods may be used, but an amplicon NGS method may be preferred.

In step S420, reads having the same insertion sequence may be selected from among the acquired reads based on a reference sequence. The reference sequence refers to a sequence for a conventional well-known target region, and the reference sequence and the acquired reads may be sorted in various methods, and sort alignment of reads by insertion size may be performed.

Also, in S420, reads having soft-clipped bases may be selected, and the meaning of soft-clipped bases has been described above. To derive the soft-clipped bases, sort alignment of reads by base may be performed.

In step S430, a region including a part or all of the sequence of the soft-clipped bases or/and insertion sequence of the selected reads may be selected as a SEED.

In step S440, ITD may be analyzed using the acquired SEED, and the analysis may count the number of ITDs and derive VAF by dividing the number of ITDs by the total number of ITDs. Based on VAF, it is possible to predict a clinical condition of a patient, and for example, it is possible to provide information for diagnosing a disease of a patient, predict the prognosis of a specific patient, or provide information for predicting a therapeutic response of a patient.

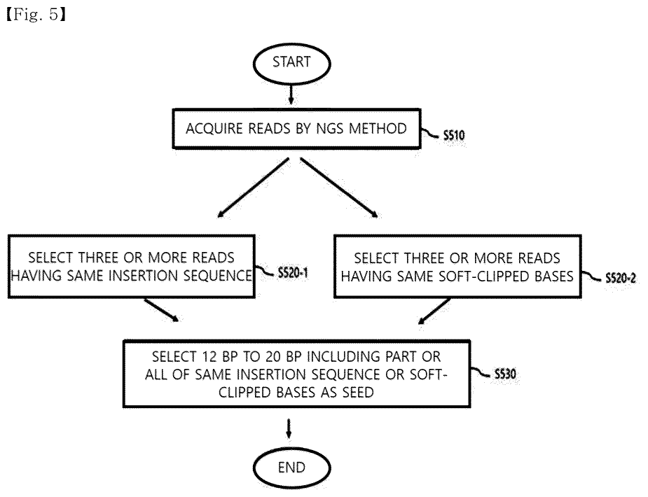

FIG. 5 is a flowchart for describing a method for deriving a SEED according to an exemplary embodiment in more detail.

Step S510 is a method of acquiring reads by an NGS method, and more specifically, may acquire read information by an amplicon NGS method.

Step S520 is a step of selecting specific reads, and may select reads when three or more reads have the same insertion sequence (S520-1) and/or when three or more reads have the same sequence of soft-clipped bases (S520-2). The steps may be performed independently or simultaneously.

Step S530 is a step of determining the SEED, and may determine sequences near soft-clipped bases of three or more reads including the same sequence of soft-clipped bases as the SEED, and more specifically, determine a sequence adjacent to the brick point of the soft-clipped segment, i.e., the 3′ or 5′ end of the soft-clipped base as the SEED. The SEED may include an adjacent sequence from the 3′ or 5′ end, and the SEED may include a part or all of the soft-clipped bases, and the sequence length may be 12 bp to 20 bp.

In addition, sequences near the insertion sequence of three or more reads including the same insertion sequence may be set as a SEED, and more particularly, the SEED includes all or a part of the insertion sequence and an adjacent sequence from the 3′ or 5′ end of the insertion sequence, but the sequence length including the adjacent sequence from the 3′ or 5′ end of the insertion sequence may be 12 bp to 20 bp. That is, the SEED includes a part or all of the insertion sequence, but includes an adjacent sequence of the insertion sequence.

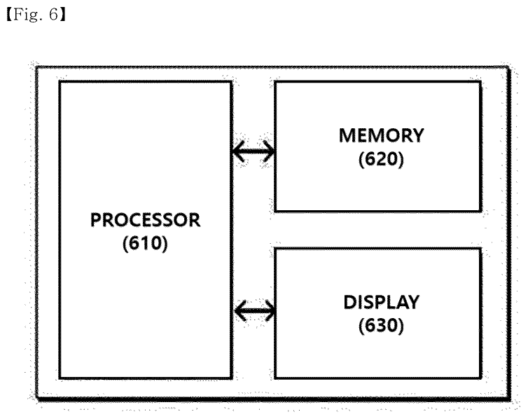

FIG. 6 is a block diagram of an apparatus 600 for deriving a SEED according to an exemplary embodiment.

Referring to FIG. 6, the apparatus 600 may include a processor 610, a memory 620, and a display 630. According to the apparatus 600 in the exemplary embodiments, the processor 610 may operate. However, components of the apparatus 600 for deriving the SEED according to an exemplary embodiment are not limited to the above-described example. In another exemplary embodiment, the apparatus 600 for deriving the SEED may include more or fewer components than the components described above.

The processor 610 may acquire information about reads for an arbitrary sequence by an NGS analysis method, select reads having the same insertion sequence from among the acquired reads based on a reference sequence; or/and b) select reads having the same soft-clipped bases, and select, as a SEED, a region including a part or all of the sequence of the soft-clipped bases of the selected reads or/and the insertion sequence thereof.

In the step of selecting the reads, the processor may select the reads having the same insertion sequence when three or more reads have the same insertion sequence and select the reads when three or more reads have the same sequence of the soft-clipped bases.

The region including the sequence of the soft-clipped bases includes an adjacent sequence from the 3′ or 5′ end of the soft-clipped base, and the sequence length including the adjacent sequence from the 3′ or 5′ end of the soft-clipped base may be 12 bp to 20 bp.

The region including the insertion sequence includes an adjacent sequence from the 3′ or 5′ end of the insertion sequence, and the sequence length including the adjacent sequence from the 3′ or 5′ end of the insertion sequence may be 12 bp to 20 bp.

The memory 620 may store information about the reads and information about the reference sequence and the SEED.

The display 630 may display information about a SEED or ITD, disease prognosis, etc., and may provide a DB descriptive text for the SEED together as described above in FIG. 5.

The apparatus according to the present disclosure may include a processor, a memory storing and executing program data, a permanent storage such as a disk drive, a communication port communicating with an external device, a user interface device such as a touch panel, a key, and a button, and the like. Methods implemented by software modules or algorithms may be stored on computer readable recording media as computer-readable codes or program instructions that may be executed on the processor. Here, the computer readable recording media include magnetic storage media (e.g., read-only memory (ROM), random-access memory (RAM), floppy disk, hard disk, etc.), optical reading media (e.g., CD-ROM, and digital versatile disc (DVD)), and the like. The computer readable recording media may be stored and executed with codes which may be distributed in computer systems connected via a network and readable by a computer in a distribution method. The media are readable by a computer, stored in the memory, and may be executed in the processor.

All documents including publications, patent applications, patents, etc. cited in the present disclosure are illustrated by combining each cited reference individually and specifically, or combined with the present disclosure in the same manner as those combined and indicated in the present disclosure as a whole.

In order to understand the present disclosure, reference numerals are given in the preferred exemplary embodiments shown in the drawings, specific terms have been used to describe the exemplary embodiments of the present disclosure, but the present disclosure is not limited by the specific terms, and the present disclosure may include all components commonly conceived by those skilled in the art.

The present disclosure may be represented by functional block configurations and various processing steps. These functional blocks may be implemented as various numbers of hardware or/and software configurations for executing specific functions. For example, the present disclosure may adopt IC configurations, such as a memory, a processing, a logic, a look-up table, and the like, which may execute various functions by control of one or more microprocessors or other control devices. The components of the present disclosure include various algorithms implemented in combination of a data structure, processes, routines, or other programming configurations, like being executed by software programming or software elements to be implemented by a programming or scripting language such as C, C++, Java, assembler, and the like. Functional aspects may be implemented as algorithms executed in one or more processors. In addition, the present disclosure may adopt the related art for electronic environment configuration, signal processing, and/or data processing. The terms “mechanism”, “element”, “means”, and “configuration” may be widely used and are not limited to mechanical and physical configurations. The terms may include the meaning of a series of processes (routines) of software in conjunction with a processor or the like.

The specific implementations described in the present disclosure are exemplary embodiments, and do not limit the scope of the present disclosure in any way. For brevity of the specification, descriptions of conventional electronic configurations, control systems, software, and other functional aspects of the systems may be omitted. In addition, the connection or connection members of lines between the components illustrated in the drawings exemplarily represent functional connections and/or physical or circuit connections, and may be illustrated as various functional connections, physical connections, or circuit connections that may be replaced or added in an actual device. In addition, unless specifically stated, such as “essential” or “important”, components may not necessarily be required for the application of the present disclosure.

As used in the present disclosure (especially, in the appended claims), the use of the term “the” and similar instruction terms thereto may correspond to both singular and plural references. Also, it should be understood that any numerical range recited herein is intended to include all sub-ranges subsumed therein (unless expressly indicated otherwise) and therefore, the disclosed numeral ranges include every individual value between the minimum and maximum values of the numeral ranges. Finally, If there is no explicit description or contradiction of the order of steps constituting the method according to the present disclosure, the steps may be performed in any suitable order. In other words, the present disclosure is not necessarily limited to the order in which the individual steps are recited. All examples described herein or the indicative terms thereof (for example, etc.) used herein are merely to describe the present disclosure in more detail. Therefore, it should be understood that the scope of the present disclosure is not limited to the exemplary embodiments or indicative terms unless limited by the appended claims. Also, it should be apparent to those skilled in the art that various modifications, combinations, and alternations may be made depending on design conditions and factors within the scope of the appended claims or equivalents thereof.

1. A method for deriving a sequence for analyzing internal tandem duplication (ITD) in an NGS method, the method comprising:
1) acquiring reads by an NGS method;
2) a) selecting reads having the same insertion sequence from among the acquired reads based on a reference sequence; or/and b) selecting reads having the same soft-clipped bases; and
3) selecting, as a SEED, a region including a part or all of the sequence of the soft-clipped bases of the selected reads or/and the insertion sequence thereof.

2. The method of claim 1, wherein in the selecting of the reads in step 2), when three or more reads have the same insertion sequence, the reads having the same insertion sequence are selected.

3. The method of claim 1, wherein in the selecting of the reads in step 2), when three or more reads have the same sequence of the soft-clipped bases, the reads are selected.

4. The method of claim 1, wherein in step 3), a region including the sequence of the soft-clipped bases includes an adjacent sequence from the 3′ or 5′ end of the soft-clipped base,
wherein the sequence length including the adjacent sequence from the 3′ or 5′ end of the soft-clipped base is 12 bp to 20 bp.

5. The method of claim 1, wherein in step 3), a region including the insertion sequence includes an adjacent sequence from the 3′ or 5′ end of the insertion sequence, wherein the sequence length including the adjacent sequence from the 3′ or 5′ end of the insertion sequence is 12 bp to 20 bp.

6. The method of claim 1, wherein the NGS method is an amplicon-based NGS method.

7. A method for analyzing internal tandem duplication (ITD) in an NGS method, the method comprising:
1) acquiring reads by an NGS method;
2) a) selecting reads having the same insertion sequence from among the acquired reads based on a reference sequence; or/and b) selecting reads having the same soft-clipped bases;
3) selecting a region including a part or all of the sequence of the soft-clipped bases or/and insertion sequence of the selected reads as a SEED; and
4) analyzing sequences matching the SEED with respect to reads acquired by an arbitrary NGS method using the selected SEED as a query.

8. The method of claim 7, wherein the analyzing of step 4) is a step of counting the number of matched sequences.

9. An apparatus for deriving a sequence for analyzing internal tandem duplication (ITD) in next generation sequence (NGS) analysis, the apparatus comprising:
a processor configured to acquire information about reads for an arbitrary sequence by an NGS analysis method, select reads having the same insertion sequence from among the acquired reads based on a reference sequence; or/and b) select reads having the same soft-clipped bases, and select, as a SEED, a region including a part or all of the sequence of the soft-clipped bases of the selected reads or/and the insertion sequence thereof;
a memory configured to store information about the reads and information about the reference sequence and the SEED; and
a display configured to display information about the derived SEED.

10. The apparatus of claim 9, wherein in the selecting of the reads, when three or more reads have the same insertion sequence, the reads having the same insertion sequence are selected.

11. The apparatus of claim 9, wherein in the selecting of the reads, when three or more reads have the same sequence of the soft-clipped bases, the reads are selected.

12. The apparatus of claim 9, wherein a region including the sequence of the soft-clipped bases includes an adjacent sequence from the 3′ or 5′ end of the soft-clipped base,
wherein the sequence length including the adjacent sequence from the 3′ or 5′ end of the soft-clipped base is 12 bp to 20 bp.

13. The apparatus of claim 9, wherein a region including the insertion sequence includes an adjacent sequence from the 3′ or 5′ end of the insertion sequence, wherein the sequence length including the adjacent sequence from the 3′ or 5′ end of the insertion sequence is 12 bp to 20 bp.
